(19) United States
(12) Patent Application Publication
(10) Pub. No.: US 2025/0040827 A1
(43) Pub. Date: Feb. 6, 2025

(54) MULTICHANNEL ENDORECTAL COIL FOR PROSTATE MRI, SYSTEM, AND WORKING METHOD

(71) Applicant: SHENZHEN INSTITUTES OF ADVANCED TECHNOLOGY CHINESE ACADEMY OF SCIENCES, Shenzhen (CN)

(72) Inventors: Ye LI, Shenzhen (CN); Zhiguang MO, Shenzhen (CN); Xiaoping ZHANG, Shenzhen (CN); Huageng LIANG, Shenzhen (CN); Chao LUO, Shenzhen (CN); Qiaoyan CHEN, Shenzhen (CN); Changjun TIE, Shenzhen (CN); Qi CAO, Shenzhen (CN); Wen XIAO, Shenzhen (CN)

(73) Assignee: SHENZHEN INSTITUTES OF ADVANCED TECHNOLOGY CHINESE ACADEMY OF SCIENCES, Shenzhen (CN)

(21) Appl. No.: 18/921,953

(22) Filed: Oct. 21, 2024

Related U.S. Application Data
(63) Continuation of application No. PCT/CN2023/130795, filed on Nov. 9, 2023.

(30) Foreign Application Priority Data
Nov. 10, 2022 (CN) 202211408300.4

(51) Int. Cl. A61B 5/055; A61B 5/00; G01R 33/34; G01R 33/36

(52) U.S. Cl. CPC A61B 5/055; A61B 5/4381; G01R 33/34084; G01R 33/365

(57) ABSTRACT

A multichannel endorectal coil for prostate MRI, a system, and a working method. The coil comprises a support body provided with a winding curved surface, a plurality of first endorectal coils (1, 2, 3) wrapped around the surface of the support body, and a second endorectal coil stacked on the plurality of first endorectal coils (1, 2, 3); wherein two adjacent first endorectal coils (1, 2, 3) are decoupled by means of partial overlapping, and two non-adjacent first endorectal coils (1, 2, 3) are decoupled by providing a shared capacitor (5); the second endorectal coil comprises a first coil section and a second coil section which are in intersecting connection with one another, the first coil section and the second coil section are arranged symmetrically, and no electrical connection exists at the intersection thereof. The invention increases the number of channels, and further achieves better high-resolution imaging capabilities.

### CROSS-REFERENCE TO RELATED APPLICATIONS

The application claims priority to Chinese patent application No. 202211408300.4, filed on Nov. 10, 2022, the entire contents of which are incorporated herein by reference.

### TECHNICAL FIELD

The present invention belongs to the technical field of magnetic resonance imaging, and particularly relates to a multichannel endorectal coil for prostate MRI, a system, and a working method.

### BACKGROUND

A magnetic resonance imaging technology is widely applied in the field of medical imaging, and is of great significance in acquiring medical images and using the medical images for subsequent disease diagnosis. For example, during the prostate cancer diagnosis, high-resolution imaging plays an important role in improving the accuracy of a diagnosis result. A signal-to-noise ratio is a core parameter considered in the design of a magnetic resonance coil. A high signal-to-noise ratio means higher resolution and contrast, and further means better imaging quality.

A magnetic resonance signal rapidly decays as a distance increases, and a greater distance between a Region Of Interest (ROI) and the coil indicates a weaker magnetic resonance signal obtained. Therefore, a higher signal-to-noise ratio of the coil in the ROI indicates a stronger capability of the coil to obtain high-resolution imaging in this region. Tissue such as the prostate is located deep in the body, far from a conventional surface coil, which usually makes it difficult to obtain high-resolution imaging. Because an endorectal coil is inserted into rectum of a subject and lies close to prostate when used, a higher signal-to-noise ratio than that of the surface coil can be obtained, and thus a stronger high-resolution imaging capability can be obtained.

In the prior art, the patent (patent publication No. CN101482600A, entitled “RECEIVING APPARATUS OF MAGNETIC RESONANCE IMAGING SYSTEM”) discloses the following technical feature: in a case that a surface coil is used, increasing the number of loops can improve a signal-to-noise ratio when the loops are all in a same plane. However, when the loops are not in the same plane, the greater number of loops indicates severer interference between the loops, and since the space inside the rectum is limited, it is difficult to increase the number of loops while maintaining a low degree of interference. The original endorectal coil includes a single loop. The patent (patent publication No. CN1802123A, entitled “SYSTEM AND METHOD FOR ACQUIRING IMAGES AND SPECTRA OF INTRACAVITY STRUCTURE USING 3.0 TESLA MAGNETIC RESONANCE SYSTEM”) discloses the following technical feature: the original endorectal coil successfully obtains a better imaging effect than a commercial surface coil, but still cannot meet a high-resolution imaging requirement. In order to improve performance of the coil, the number of loops of the endorectal coil is increased to two. In order to solve the problem of mutual interference caused after the number of loops is increased, an orthogonal microstrip line is used as a second channel in existing research (refer to the document: Arteaga de Castro C S, Van Den Bergen B, Luijten P R, et al. Improving SNR and B1 transmit field for an endorectal coil in 7 T MRI and MRS of prostate cancer [J]. Magnetic resonance in medicine, 2012, 68(1): 311-318.), and a method in which two loops are perpendicular to each other is used in other research (refer to the document: Ertürk M A, Tian J, Van de Moortele PF, et al. Development and evaluation of a multichannel endorectal RF coil for prostate MRI at 7T in combination with an external surface array [J]. Journal of Magnetic Resonance Imaging, 2016, 43 (6): 1279-1287.) as well as the prior patent (patent publication No. 104541177A, entitled “ENDORECTAL PROSTATE COIL WITH OPEN ACCESS FOR SURGICAL INSTRUMENTS”). Thereafter, the number of loops of the coil is difficult to continue to increase.

Therefore, how to reduce the interference between the loops while increasing the number of loops to meet the high-resolution imaging requirement becomes an urgent technical problem to be solved.

### SUMMARY

The present invention intends to provide a multichannel endorectal coil for prostate MRI, a system, and a working method, to solve the technical problem in the prior art that the design of an endorectal coil cannot meet a high-resolution imaging requirement of a magnetic resonance system.

In order to achieve the above objective, the following technical solutions are used in the present invention.

A first aspect provides a multichannel endorectal coil for prostate MRI, comprising a support body provided with a wire-wound curved surface, a plurality of first endorectal coils wound on a surface of the support body in a wrapping manner, and a second endorectal coil superposed on the plurality of first endorectal coils, wherein

decoupling between two adjacent first endorectal coils is achieved by means of partial overlapping, and decoupling between two non-adjacent first endorectal coils is achieved by providing a shared capacitor; and the second endorectal coil comprises a first coil section and a second coil section that are connected in an intersecting manner, wherein the first coil section and the second coil section are symmetrically arranged, and no electrical connection exists at the intersection thereof.

Based on the above disclosure, in the present invention, a plurality of first endorectal coil channels or loops are arranged, and the second endorectal coil is superposed on the plurality of first endorectal coils, thereby increasing the number of channels, improving a signal-to-noise ratio, and achieving better high-resolution imaging capabilities and image quality; the second endorectal coil is arranged as the first coil section and the second coil section that are connected in the intersecting manner and are symmetrically arranged, and is superposed on the first endorectal coils, thereby increasing the density of coils, and relative positions of a loop of the second endorectal coil and the first endorectal coils are adjusted to solve a coupling problem caused by the increase of the number of channels; and the shared capacitor is arranged between the non-adjacent first endorectal coils for decoupling to reduce interference between the coils.

In a possible design, three-channel first endorectal coils that are sequentially arranged are comprised, wherein the first endorectal coil as a first channel and the first endorectal coil as the second channel partially overlap, the first endorectal coil as the second channel and the first endorectal coil as a third channel partially overlap, and at least one shared capacitor is arranged between the first endorectal coil as the first channel and the first endorectal coil as the third channel.

Based on the above disclosure, in the present invention, the first endorectal coils are arranged as three channels, which increases the number of channels of the coil compared with the prior art, and decoupling is performed by making adjacent coils partially overlap and sharing a capacitor between non-adjacent coils, thereby improving the signal-to-noise ratio of the coil, and reducing mutual interference between the coils.

In a possible design, one shared capacitor is arranged between the first endorectal coil as the first channel and the first endorectal coil as the third channel.

Based on the above disclosure, preferably, the number of shared capacitors arranged in the present invention is not limited to one, and a plurality of parallel-connected shared capacitors are arranged based on requirements, to match decoupling requirements between the non-adjacent coils.

In a possible design, two-channel first endorectal coils that are sequentially arranged are comprised, wherein the first endorectal coil as a first channel and the first endorectal coil as a second channel partially overlap.

Based on the above disclosure, in the present invention, the first endorectal coils are arranged as two channels, which increases the number of channels of the coil compared with the prior art, and decoupling is performed by making adjacent coils partially overlap and sharing a capacitor between non-adjacent coils, thereby improving the signal-to-noise ratio of the coil, and reducing mutual interference between the coils.

In a possible design, each first endorectal coil has the same structure.

Based on the above disclosure, in the present invention, structures of the three-channel first endorectal coils or the two-channel first endorectal coils are set to the same, so that the same circuit loops are formed, and the coils are uniformly wound on the support body.

In a possible design, after the first coil section and the second coil section are connected in the intersecting manner, the whole second endorectal coil is in a shape of FIG. 8.

Based on the above disclosure, the whole second endorectal coil is arranged in a shape of figure 8, so that the magnetic fields of the two coil sections of the coil are the same in magnitudes and opposite in directions, and thus a natural decoupling function exists between the second endorectal coil and each first endorectal coil when the second endorectal coil is superposed on the first endorectal coils.

In a possible design, the first coil section is superposed on a surface of a first region of each first endorectal coil, and the second coil section is superposed on a surface of a second region of each first endorectal coil.

In a possible design, the support body is arranged as a cylinder-like body with a part of a structure cut off along a length direction of the cylinder.

Based on the disclosure, in the present invention, a shape of a cylinder-like body with a D-shaped cross section is formed, so that the diameter of the cylinder can be increased as much as possible while the total volume of an insertion part of the coil remains unchanged, which in turn helps increase a loop diameter of each coil channel. The increase of the loop diameter also helps improve a signal-to-noise ratio of a target region (for example, a prostate region), thereby improving image quality of magnetic resonance imaging.

A second aspect provides a magnetic resonance imaging system, comprising the multichannel endorectal coil for prostate MRI as set forth in any possible design of the first aspect.

A third aspect provides a working method of the multichannel endorectal coil for prostate MRI as set forth in any possible design of the first aspect, comprising:
inserting the multichannel endorectal coil for prostate MRI into rectum of a subject, and establishing a connection between an endorectal coil and a magnetic resonance control unit; and
sending a control signal to the endorectal coil through the magnetic resonance control unit, so that the endorectal coil sends a radio frequency signal to a region of interest, to implement magnetic resonance signal detection of the region of interest, wherein the region of interest comprises prostate.

### BRIEF DESCRIPTION OF DRAWINGS

FIG. 1 is a schematic diagram of a three-dimensional structure of three-channel first endorectal coils according to an embodiment of the present invention;

FIG. 2 is a schematic diagram of a three-dimensional structure of a second endorectal coil according to an embodiment of the present invention;

FIG. 3 is a schematic diagram of a three-dimensional structure obtained after the second endorectal coil is superposed on the three-channel first endorectal coils according to an embodiment of the present invention;

FIG. 4 is a schematic diagram of a planar unfolded structure of the three-channel first endorectal coils according to an embodiment of the present invention;

FIG. 5 is a schematic diagram of a planar unfolded structure of the second endorectal coil according to an embodiment of the present invention;

FIG. 6 is a schematic diagram of a planar unfolded structure obtained after the second endorectal coil is superposed on the three-channel first endorectal coils according to an embodiment of the present invention;

FIG. 7 is a schematic diagram of a three-dimensional structure obtained after the second endorectal coil is superposed on two-channel first endorectal coils according to an embodiment of the present invention; and

FIG. 8 is a schematic diagram of a three-dimensional structure obtained after the second endorectal coil is superposed on the two-channel first endorectal coils according to an embodiment of the present invention.

### DETAILED DESCRIPTION OF THE EMBODIMENTS

In order to describe the technical solutions in the embodiments of the present invention or in the prior art more clearly, the following briefly describes the present invention with reference to the descriptions of the accompanying drawings and the embodiments or the prior art. It is clear that the accompanying drawings in the following description show merely some embodiments of the present invention, and those of ordinary skill in the art may still derive other drawings from these accompanying drawings without creative efforts. It should be noted herein that the description of the embodiments is provided to help understanding of the present invention, and the present invention is not limited thereto.

### Embodiments

In order to solve the technical problem in the prior art that the design of an endorectal coil cannot meet a high-resolution imaging requirement of a magnetic resonance system, in the present invention, a plurality of, for example, two or three, first endorectal coil channels or loops are arranged, and a second endorectal coil is superposed on the plurality of first endorectal coils, thereby increasing the number of channels, improving a signal-to-noise ratio, and achieving better high-resolution imaging capabilities and image quality; the second endorectal coil is arranged as a figure-8-shaped loop and superposed on the first endorectal coils, thereby increasing the density of coils, and relative positions of the loop of the second endorectal coil and the first endorectal coils are adjusted to solve a coupling problem caused by the increase of the number of channels; and a shared capacitor is arranged between non-adjacent first endorectal coils for decoupling to reduce interference between the coils.

The multichannel endorectal coil for prostate MRI provided in the embodiments of the present application is described in detail below.

It should be noted that, in the embodiments of the present application, transrectal means that the coil enters through rectum, and the prostate coil means that the coil is used to image prostate tissue. Because an endorectal coil is inserted into rectum of a patient and lies close to prostate when used, the multichannel endorectal coil for prostate MRI in the embodiments of the present application is mainly used in a magnetic resonance imaging system for acquiring magnetic resonance signals of prostate-related tissue. Certainly, it may be understood that the multichannel endorectal coil for prostate MRI in the embodiments of the present application can also be used in a magnetic resonance imaging system for acquiring magnetic resonance signals of tissue such as rectum. This is not limited herein.

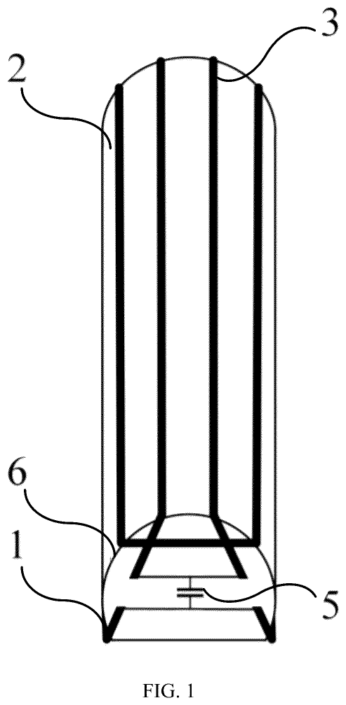

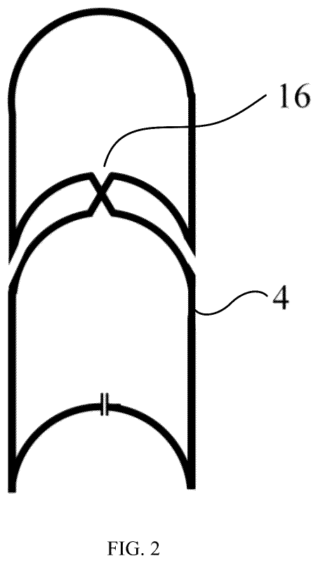

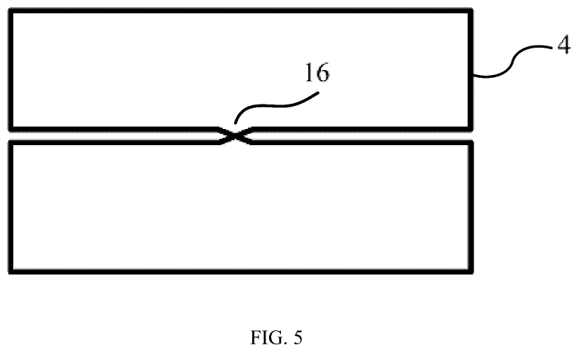

As shown in FIG. 1 to FIG. 8, a multichannel endorectal coil for prostate MRI in the embodiments of the present application includes a support body provided with a wire-wound curved surface, a plurality of first endorectal coils wound on a surface of the support body in a wrapping manner, and a second endorectal coil superposed on the plurality of first endorectal coils.

Decoupling between two adjacent first endorectal coils is achieved by means of partial overlapping, and decoupling between two non-adjacent first endorectal coils is achieved by providing a shared capacitor. The second endorectal coil includes a first coil section and a second coil section that are connected in an intersecting manner, where the first coil section and the second coil section are symmetrically arranged, and no electrical connection exists at the intersection thereof.

Because the key of the coil to obtain a high signal-to-noise ratio is to integrate as many channels as possible in a region close to an imaging target while ensuring the minimum mutual interference between the channels, an embodiment of the present application provides a multichannel endorectal coil structure, in which each coil channel corresponds to one loop, and a plurality of coil channels are integrated in a same direction, so that stronger magnetic resonance signals can be received in a region close to a target, for example, prostate, that is, the design of the endorectal coil enables the coil structure to have a high signal-to-noise ratio, and thus have a better high-resolution imaging capability.

Specifically, in the embodiment of the present application, after two adjacent coils (A and B) are overlapped, magnetic induction lines in a range of a loop of one coil A cancel each other out. In this case, when a current exists in a loop of the other coil B, no current or less current is induced in the loop of the coil A, thereby achieving a decoupling purpose. In addition, distribution of the magnetic induction lines is uneven, that is, the magnetic induction lines in the coil B are dense, and the magnetic induction lines outside the coil B (namely a non-overlapping region of the coil A) are sparse. Therefore, in order to enable the total magnetic induction lines in the coil A to exactly cancel each other out, an overlapping area is less than a half of an area of the loop of the coil A, while the special structure of intersecting connection and symmetrical arrangement of the second endorectal coil makes directions of currents induced by the first coil section and the second coil section exactly opposite; and in order to enable the total magnetic induction lines exactly cancel each other out, when the second endorectal coil overlaps loops of the plurality of first endorectal coils, the magnetic induction lines passing through the first coil section of the second endorectal coil should be exactly equal to the magnetic induction lines passing through the second coil section thereof, and therefore the first coil section and the second coil section are required to be symmetrically arranged. In this case, the second endorectal coil overlaps each first endorectal coil, which achieves a natural decoupling effect.

Preferably, the first endorectal coils in the embodiment of the present application are arranged as three-channel coils or two-channel coils. Certainly, it may be understood that, in other embodiments, the number of coils can also be appropriately increased while ensuring that the interference between the coils is low, to acquire as many stronger magnetic resonance signals as possible in a ROI.

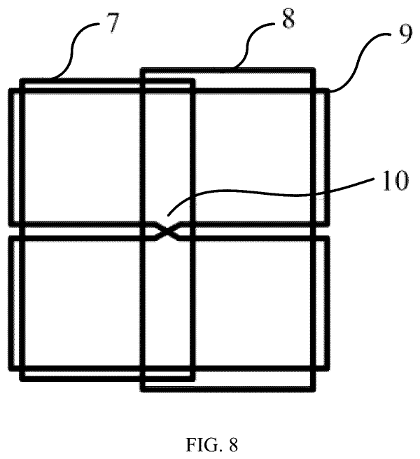

In a specific implementation, after the first coil section and the second coil section are connected in the intersecting manner, the whole second endorectal coil is in a shape of figure 8. More preferably, the first coil section is superposed on a surface of a first region of each first endorectal coil, and the second coil section is superposed on a surface of a second region of each first endorectal coil. Specifically, the first coil section and the second coil section are arranged as two mutually intersecting approximate rectangles in a planar unfolded state, and an intersecting position is a central position of the whole second endorectal coil, to ensure that the magnetic fields of the first coil section and the second coil section are same in magnitudes and opposite in directions, which achieves the natural decoupling effect. Certainly, it may be understood that the above arrangement of the first coil section and the second coil section as the approximate rectangles is only one design of the embodiment of the present application, mainly for meeting the requirement of winding on the curved surface. In other embodiments, the coil sections may alternatively be arranged in other shapes, such as a sector and an ellipse. This is not limited herein.

For example, as shown in FIG. 1 to FIG. 6, in a specific embodiment, three-channel first endorectal coils that are sequentially arranged are included, where the first endorectal coil as a first channel and the first endorectal coil as a second channel partially overlap, the first endorectal coil as the second channel and the first endorectal coil as a third channel partially overlap, and at least one shared capacitor is arranged between the first endorectal coil as the first channel and the first endorectal coil as the third channel. Preferably, one shared capacitor is arranged between the first endorectal coil as the first channel and the first endorectal coil as the third channel.

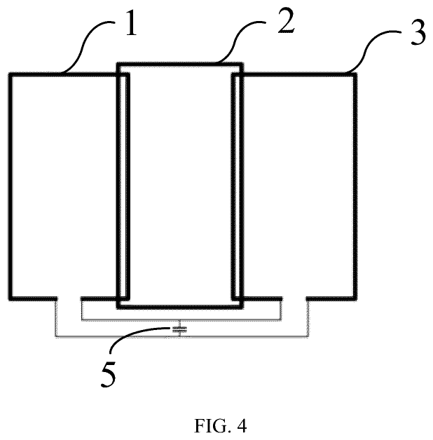

For example, as shown in FIG. 1 to FIG. 3, the first endorectal coil 1 as the first channel and the first endorectal coil 2 as the second channel are included, and an overlap exists at adjacent parts of the first endorectal coil 1 as the first channel and the first endorectal coil 2 as the second channel. Similarly, an overlap exists at adjacent parts of the first endorectal coil 3 as the third channel and the first endorectal coil 2 as the second channel, so that decoupling between adjacent coils can be achieved to reduce mutual interference. In addition, one shared capacitor 5 is arranged between the first endorectal coil 1 as the first channel and the first endorectal coil 3 as the third channel, so that decoupling between the first endorectal coil 1 as the first channel and the first endorectal coil 3 as the third channel is achieved, and interference between the first endorectal coil 1 as the first channel and the first endorectal coil 3 as the third channel is reduced. In addition, the second endorectal coil is arranged in a shaped of figure 8, so that the second endorectal coil can be uniformly covered on the three-channel first endorectal coils, upper and lower parts of the figure-8-shaped coil are respectively superposed on symmetrical upper and lower parts of channels 1, 2 and 3, and no electrical connection exists at the intersection 16 of the figure-8-shaped channel. Because magnetic fields of the upper and lower parts of the figure-8-shaped coil are same in magnitudes and opposite in directions, and magnetic fields of the upper and lower parts of the channels 1, 2 and 3 are same in magnitudes and same in directions, a fourth channel (namely the second endorectal coil) and the channels 1, 2 and 3 all have natural decoupling performance, and therefore the mutual interference between the channels can be effectively reduced. Finally, the coil as the channel 4 is wound on the surface of the support body 6 in a wrapping manner, so that the whole endorectal coil is designed into a curved surface, and thus can be adapted to tissue such as rectum to acquire the magnetic resonance signals. In addition, as shown in FIG. 4 to FIG. 6, the schematic diagram of each coil in the planar unfolded state can be clearly seen. It can be seen that the three-channel first endorectal coils are preferably three rectangular coils which are sequentially overlapped, and an opening is additionally provided between the first channel and the third channel for mutual connection, and then a shared capacitor is connected in parallel between connection lines for decoupling.

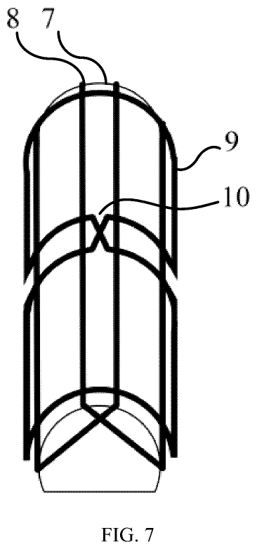

For example, as shown in FIG. 7 to FIG. 8, two-channel first endorectal coils that are sequentially arranged are included, where the first endorectal coil 7 as a first channel and the first endorectal coil 8 as a second channel partially overlap, so that decoupling between adjacent coils can be achieved to reduce mutual interference. In addition, the second endorectal coil is arranged in a shaped of figure 8, so that the second endorectal coil can be uniformly covered on the two-channel first endorectal coils, upper and lower parts of a channel 9 of the figure-8-shaped coil are respectively superposed on symmetrical upper and lower parts of channels 7 and 8, and no electrical connection exists at the intersection 10 of the figure-8-shaped channel. Because magnetic fields of the upper and lower parts of the figure-8-shaped coil are same in magnitudes and opposite in directions, and magnetic fields of the upper and lower parts of the channels 7 and 8 are same in magnitudes and same in directions, a ninth channel (namely the second endorectal coil) and the channels 7 and 8 all have natural decoupling performance, and therefore the mutual interference between the channels can be effectively reduced. Finally, the coil as the channel 3 is wound on the surface of the support body in a wrapping manner, so that the whole endorectal coil is designed into a curved surface, and thus can be adapted to tissue such as rectum to acquire the magnetic resonance signals.

In a specific implementation, in the embodiment of the present application, each first endorectal coil preferably has the same structure. For example, structures of the three-channel first endorectal coils or the two-channel first endorectal coils are set to the same, so that the same circuit loops are formed, and the coils are uniformly wound on the support body.

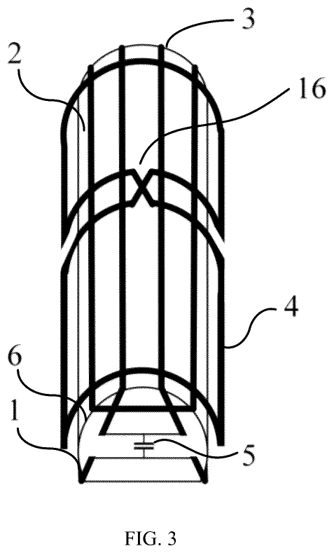

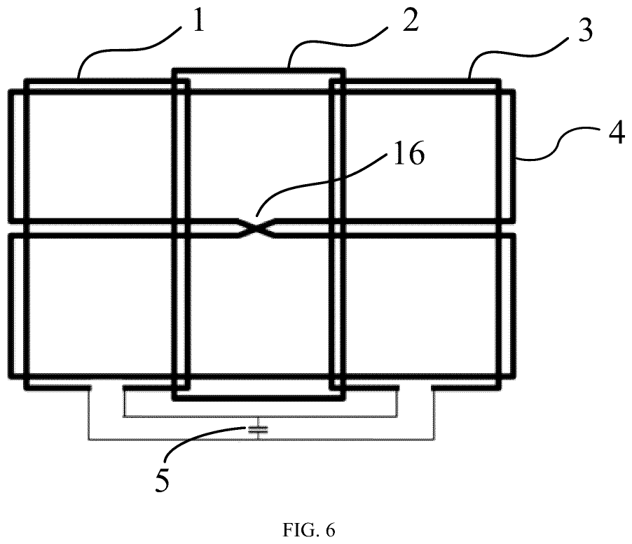

In a specific implementation, the support body is arranged as a cylinder-like body with a part of a structure cut off along a length direction of the cylinder. For example, FIG. 1, FIG. 3, and FIG. 6 show the structure of the support body in the embodiment of the present application. It can be learned from the drawings that the support body is in a shape of a cylinder-like body with a D-shaped cross section that is formed by uniformly cutting off a part of a structure of a cylinder along a plane in a length direction of the cylinder, so that the diameter of the cylinder can be increased as much as possible while the total volume of an insertion part of the coil remains unchanged, which in turn helps increase a loop diameter of each coil channel. The increase of the loop diameter also helps improve a signal-to-noise ratio of a target region (for example, a prostate region), thereby improving image quality of magnetic resonance imaging. Certainly, it may be understood that the support body in the embodiment of the present application is not limited to the structure given in the above example, and may alternatively be set to a cylinder with an elliptical cross section or other approximately circular cross sections, thereby providing a support body for the coil to be wound in the wrapping manner. This is not specifically limited herein.

Based on the above disclosure, the embodiment of the present application includes a support body provided with a wire-wound curved surface, a plurality of first endorectal coils wound on a surface of the support body in a wrapping manner, and a second endorectal coil superposed on the plurality of first endorectal coils. Decoupling between two adjacent first endorectal coils is achieved by means of partial overlapping, and decoupling between two non-adjacent first endorectal coils is achieved by providing a shared capacitor. The second endorectal coil includes a first coil section and a second coil section that are connected in an intersecting manner, where the first coil section and the second coil section are symmetrically arranged, and no electrical connection exists at the intersection thereof. That is, in the present invention, a plurality of, for example, two or more, first endorectal coil channels or loops are arranged, and a second endorectal coil is superposed on the plurality of first endorectal coils, thereby increasing the number of channels, improving a signal-to-noise ratio, and achieving better high-resolution imaging capabilities and image quality; the second endorectal coil is arranged as a figure-8-shaped loop and superposed on the first endorectal coils, thereby increasing the density of coils, and relative positions of the loop of the second endorectal coil and the first endorectal coils are adjusted to solve a coupling problem caused by the increase of the number of channels; and a shared capacitor is arranged between non-adjacent first endorectal coils for decoupling to reduce interference between the coils.

A second aspect provides a magnetic resonance imaging system, including the multichannel endorectal coil for prostate MRI as set forth in any possible design of the first aspect.

A third aspect provides a working method of the multichannel endorectal coil for prostate MRI as set forth in any possible design of the first aspect, including but not limited to steps S1 and S2.
S1: Insert the multichannel endorectal coil for prostate MRI into rectum of a subject, and establishing a connection between an endorectal coil and a magnetic resonance control unit.
S2: Send a control signal to the endorectal coil through the magnetic resonance control unit, so that the endorectal coil sends a radio frequency signal to a region of interest, to implement magnetic resonance signal detection of the region of interest, where the region of interest includes prostate.

Finally, it should be noted that the above descriptions are merely preferred embodiments of the present invention, but not intended to limit the present invention. Any modification, equivalent replacement, or improvement made within the spirit and principle of the present invention should be included in the protection scope of the present invention.

What is claimed is:

1. A multichannel endorectal coil for prostate MRI, comprising a support body provided with a wire-wound curved surface, a plurality of first endorectal coils wound on a surface of the support body in a wrapping manner, and a second endorectal coil superposed on the plurality of first endorectal coils, wherein
decoupling between two adjacent first endorectal coils is achieved by means of partial overlapping, and decoupling between two non-adjacent first endorectal coils is achieved by providing a shared capacitor; the second endorectal coil comprises a first coil section and a second coil section that are connected in an intersecting manner, wherein the first coil section and the second coil section are symmetrically arranged, and no electrical connection exists at the intersection thereof;
after the first coil section and the second coil section are connected in the intersecting manner, the whole second endorectal coil is in a shape of figure 8;
the first coil section is superposed on a surface of a first region of each first endorectal coil, and the second coil section is superposed on a surface of a second region of each first endorectal coil; and
the support body is arranged as a cylinder-like body with a part of a structure cut off along a length direction of the cylinder.

2. The multichannel endorectal coil for prostate MRI according to claim 1, comprising three-channel first endorectal coils that are sequentially arranged, wherein the first endorectal coil as a first channel and the first endorectal coil as a second channel partially overlap, the first endorectal coil as the second channel and the first endorectal coil as a third channel partially overlap, and at least one shared capacitor is arranged between the first endorectal coil as the first channel and the first endorectal coil as the third channel.

3. The multichannel endorectal coil for prostate MRI according to claim 2, wherein one shared capacitor is arranged between the first endorectal coil as the first channel and the first endorectal coil as the third channel.

4. The multichannel endorectal coil for prostate MRI according to claim 1, comprising two-channel first endorectal coils that are sequentially arranged, wherein the first endorectal coil as a first channel and the first endorectal coil as a second channel partially overlap.

5. The multichannel endorectal coil for prostate MRI according to claim 1, wherein each first endorectal coil has the same structure.

6. A magnetic resonance imaging system, comprising the multichannel endorectal coil for prostate MRI according to claim 1.

7. A working method of the multichannel endorectal coil for prostate MRI according to claim 1, comprising:
inserting the multichannel endorectal coil for prostate MRI into rectum of a subject, and establishing a connection between anendorectal coil and a magnetic resonance control unit; and
sending a control signal to the endorectal coil through the magnetic resonance control unit, so that the endorectal coil sends a radio frequency signal to a region of interest, to implement magnetic resonance signal detection of the region of interest, wherein the region of interest comprises prostate.
